(19) United States
(12) Patent Application Publication
(10) Pub. No.: US 2009/0099261 A1
(43) Pub. Date: Apr. 16, 2009

(54) Omega-3 mixtures

(75) Inventors: Stacey Jane Bell, Belmont, MA (US); Gregory Thomas Grochoski, Ada, MI (US); Raymond Gregory Jaglowski, Ada, MI (US); Marc Christopher Stover, Caledonia, MI (US)

Correspondence Address: GOODWIN PROCTER LLP;ATTN: PATENT ADMINISTRATOR, 620 Eighth Avenue, NEW YORK, NY, 10018, US

(73) Assignee: ISI Brands Inc., Grand Rapids, MI (US)

(21) Appl. No.: 12/284,996

(22) Filed: Sep. 25, 2008

Related U.S. Application Data
(60) Provisional application No. 60/995,198, filed on Sep. 25, 2007.

(51) Int. Cl. A61K 31/202; A23L 1/30; A61P 9/00; A61P 9/12; A61P 25/24; A61P 25/00; A61P 19/02

(52) U.S. Cl. 514/560; 426/72

(57) ABSTRACT

The present invention is directed to mixtures of omega-3 fatty acids, formulations of these mixtures suitable for use in a variety of dosage forms or food/beverage additives, and uses of these mixtures for preventative or therapeutic health purposes.

This application claims priority to U.S. provisional patent application Ser. No. 60/995,198, filed on Sep. 25, 2007, which is incorporated by reference in its entirety.

### BACKGROUND

Dietary intake of omega-3 fatty acids (omega-3s) is associated with a variety of therapeutic and preventative health benefits. For example, research suggests a potential benefit of dietary omega-3s in the treatment of many of the chronic diseases of aging such as depression (A. L. Stoll., Arch. Gen. Psychiatry 56:407-412 (1999); D. Volker and Jade Ng, Nutrition and Dietics 63:213-226 (2006); M. Peet and D. F. Horribon, Arch. Gen. Psychiatry 59:913-919 (2002)), senility (G. Fontani et al. Eur. J Clin. Invest. 35:691-699 (2005)), cardiovascular disease (GISSI Prevenzione Investigators, Lancet 354:447-455 (1999); Roberto Marchioli et al., Lipids 36(Supplement):S119-S126 (2001); Roberto Marchioli et al., Lancet 354:447-455 (1999); Clemens Von Schacky, et. al., Ann Intern Med, 1999; 130:554-62), blood pressure lowering (George Paschos et al., Eur J Clin Nutr, published on line, 31 Jan. 2007: doi:10.1038/sj.jcn.1602631)), and arthritis (L. Deutsch., J. Am. Coll. Nutr. 26:39-48 (2007); J. M. Kremer, Lancet Jan. 26, 1985 at 184-187; J. M. Kremer, Ann. Intern. Med. 106:497-503 (1987)). Dietary intake of certain omega-3s also appears to moderate a variety of other conditions such as attention deficit hyperactivity disorder (A. J. Richardson and P. Montgomery, Pediatrics 115:1360-1366 (2005); N. Sinn and J. Bryan, J. Dev. Behav. Pediatr. 28:82-91 (2007); Paul J. Sojgi et al., Nutrition Journal 6:16 (2001)), premenstrual syndrome and dysmenorrhea (F. Sampalis et al., Alternative Medicine Review 8:171-179 (2003)), and hyperlipidemia (Ruxandra Bunea et al., Alternative Medicine Review 9:420-428 (2004)).

Dietary omega-3s are ingested primarily from plant and marine-derived foods. Omega-3s from plant sources such as flax, chia, and echium (e.g Incromega V3 Croda) oil are typically rich in short-chain (18 carbons long) omega-3 fatty acids, while omega-3s from marine sources such as krill and fish are typically rich in long-chain (more than 18 carbons) omega-3s. Other dietary sources of omega-3s include meat (e.g., beef and chicken) and eggs, though these are not presently preferred. Omega-3s (both short- and long-chains) are essential dietary components in part because they serve as substrates for eicosanoids, which are essential biologically active compounds. Long-chain omega-3s are more efficiently converted to compounds like eicosanoids in vivo than are short chain omega-3s. Linolenic acid (LNA, 18:3 omega-3), like all other omega-3 fats, is an essential dietary component because it can supply a fat with a double-bond in the number 3 position of the fatty acid chain. The fatty acid content of chia oil includes approximately 63.8% LNA. Chia contains about 10% more LNA than does flax. Ten percent or less of ingested LNA from plant oils (e.g., chia, flax) is converted into eicosapentaenoic acid (EPA, 20:5 omega-3). Chia oil is commercially available from a number of sources.

Another plant-derived fat that contains short-chain omega-3s is echium (e.g. Incromega V3 Croda) oil, which contains about 29.5% LNA as well as about 12.6% stearidonic acid (SA, 18:4 omega-3). Dietary SA is available only from plant sources. SA is more efficiently converted to EPA than is LNA: 40% of dietary SA is converted to EPA, while only about 10% or less of dietary LNA is converted to EPA. (Michael J. James et al., Am. J. Clin. Nutr. 77:1140-45 (2003)). In contrast, standard plant oils are not as easily converted to EPA as is echium oil because standard plant oils do not contain sufficient amounts of SA. Although echium oil contains only half as much LNA as does either flax or chia oil, conversion of echium oil to the long-chain omega-3 fat EPA is substantial due to the SA content of echium oil. For each gram of echium oil, in theory about 80 mg of EPA is produced by the body - about 30 mg from LNA (echium oil contains approximately 30% LNA of which 10% is converted to EPA) and about 50 mg from SA (echium oil contains about 12.5% SA of which about 40% is converted to EPA). Echium oil is available, for example, under the trade name Incromega V3 from Croda Chemicals Europe Ltd. Health Care.

Dietary intake of long-chain omega-3s (LCO3) results primarily from consumption of marine-derived foods. The biologically important omega-3s found in krill and fish oil are EPA and docosahexaenoic acid (DHA, 22:6 omega-3). Most LCO3s contained in fish or fish oil are naturally in the form of triglycerides (TG), wherein the essential omega-3 fatty acids can be attached at the number 1, 2, or 3 position of the glycerol molecule. (Attachment at the 1-position is referred to as sn-1, attachment at the 2- position is referred to as sn-2, and attachment at the 3- position is referred to as sn-3.) In contrast, the LCO3s contained in krill oil are in the form of phospholipids (PL). In phospholipids, the sn-1 position is occupied by a phosphate ion, and so no fatty acid can attach at the 1- position. This is significant because in the digestive metabolism of TG, sn-1 and sn-3 attached fatty acids are more readily cleaved from the triglyceride, leaving the corresponding free fatty acids and the sn-2 substituted monoglyceride. The sn-2 substituted monoglyceride is preferentially absorbed over the free fatty acids. (Stacey J. Bell et al., J. Am. Diet Assoc. 97:280-286 (1997)). The probability that an omega-3 fat would appear in the sn-2 position of a TG is 33%. In contrast, the probability that an omega-3 fat would appear in the sn-2 position of a PL is 50%. Thus, there is a 50% greater chance of absorbing omega-3 fats from a PL than a TG. Fish oil is available by extraction from fish or from a variety of commercial sources. Krill oil is available by extraction from krill, or commercially from Neptune Technologies and other suppliers.

Emulsification of omega-3 containing oils prior to administration can provide a number of important benefits. First, emulsification can improve the palatability of omega-3 containing oils, leading to increased dietary intake. Second, emulsification can improve intestinal absorption of ingested omega-3s. In one study, absorption of both short-chain and long-chain omega-3s was significantly improved (i.e., appeared in the blood at a higher concentration after feeding) when compared to absorption of the same oils not emulsified. This study suggests that emulsification can generally be expected to result in about 60% better absorption. (Iveta Garaiova et al., Nutrition Journal 6:4 (2007)). Third, emulsification can enhance the rate of clearance of blood triglycerides and increase the rate of EPA incorporation into the phospholipids (C. M. Simoens et al., Am. J. Clin. Nutr. 2008, 88:282-288). Intravenous emulsified fish oil given combined with medium-chain triglycerides (MCTs) and another long-chain triglyceride (LCT) resulted in greater clearance of plasma triglycerides and rapid incorporation of EPA, compared to a non-emulsified mixture of an MCT and LCT oil.

Because most individuals do not consume enough omega-3s to enjoy the health benefits associated with omega-3s, there is a need for dietary supplements and food additives containing omega-3s so as to improve overall health. The American diet generally contains too many omega-6s and so supplementation of the diet with omega-3s also improves the blood ratio of omega-3/omega-6 dietary fats. Excessive dietary omega-6s is thought to increase the risk of chronic diseases like heart disease, arthritis, and depression.

### SUMMARY OF THE INVENTION

The present invention is directed to mixtures of omega-3 fatty acids, formulations of these mixtures suitable for use in a variety of dosage forms or food/beverage additives, and uses of these mixtures for preventative or therapeutic health purposes.

Mixing oils from different sources, allows one to tailor the omega-3 content of the mixture so that it is beneficial for treating a particular health condition or for promoting the health of a particular class of subjects (e.g., women, pregnant women, newborns, elderly, animals), and at the same time provides for the dietary needs for other omega-3s. In particular, mixtures of omega-3s from different sources can provide EPA and DHA at levels that are beneficial for treatment of a particular health condition or for a particular subject population, while at the same time providing LNA and/or SA as building blocks for metabolic conversion to EPA.

Accordingly, the present invention provides, in one embodiment, a composition of long chain omega-3 fatty acids and short chain omega-3 fatty acids in a weight ratio of about 1:2 to about 1:10. In another embodiment, the present invention provides a composition of omega-3 containing oils, where a first component is krill oil, and a second component fish oil, flax oil, chia oil, echium oil, or mixtures thereof. In yet another embodiment, the present invention provides a composition of omega-3 containing oils, where a first component comprises fish oil, and a second component is chia oil, echium oil, or mixtures thereof.

In another embodiment, the present invention provides a formulation comprising a marine-derived omega-3 containing oil, a plant-derived omega-3 containing oil, and an excipient. In yet another embodiment, the present invention provides a formulation comprising a long chain omega-3 fatty acid and a short chain omega-3 fatty acid.

In another embodiment, the present invention provides a process for forming a composition comprising mixing at least two omega-3 fatty acids selected from the group consisting eicosapentaenoic acid, docosahexaenoic acid, linolenic acid and stearidonic acid.

In another embodiment, the present invention provides foods or beverages comprising the omega-3 compositions and formulations of the present invention.

In yet another embodiment, the present invention provides methods of increasing dietary absorption of omega-3 fatty acids by administering the omega-3 fatty acid compositions to subjects in need thereof. The present invention also provides methods of treating a variety of heath disorders by administering the omega-3 fatty acid compositions to subjects in need thereof.

### DETAILED DESCRIPTION

As used herein, the terms omega-3s, omega-3 fatty acid(s), omega-3 fat(s) and the like are meant to encompass omega-3 fatty acids as well as biologically relevant esters of these fatty acids including but not limited to triglycerides and phospholipids. These terms are also meant to encompass omega-3-containing oils (e.g., plant-derived oils, marine-derived oils, fungi-derived oils, bacteria-derived oils, and yeast-derived oils), omega-3 fatty acids substantially purified from oils, and synthetically prepared omega-3s. The omega-3 fatty acids used in this invention may take any physical form (e.g., solid, powder, liquid, emulsified mixtures, etc.).

As used herein, the names of individual omega-3 fatty acids (e.g., eicosapentaenoic acid, docosahexaenoic acid, stearidonic acid, linolenic acid) refer to the carboxylic acid and esters of the acid.

As used herein, the term fish oil is meant to encompass the oil from any fish or fish part, or blends of oils from any fish or fish part, including but not limited to cod, cod liver, menhaden, sardines, salmon, anchovy, herring, and mackerel.

As used herein, the term plant-derived means that the material was obtained from a plant or plant part, such as seed, fruit, nut, or leaf.

As used herein, the term marine-derived means that the material was obtained from a marine animal such as fish, krill, plankton, or shellfish.

The present invention features a mixture of at least two different omega-3 fatty acids. Optionally, any or all of the constituent omega-3s, or the mixture of omega-3s, or both, may be emulsified. In one embodiment, the mixture comprises at least one plant-derived omega-3 fatty acid and at least one marine-derived omega-3 fatty acid. In another embodiment, the mixture comprises at least two plant-derived omega-3 fatty acids. This embodiment is particularly useful for administration to vegetarians. In another embodiment, the mixture comprises at least two marine-derived omega-3 fatty acids. This embodiment is advantageous because both omega-3s are efficiently converted to bioactive molecules that serve as precursors to eicosanoids. In another embodiment, the mixture comprises at least one long chain omega-3 fatty acid and at least at least one short chain omega-3 fatty acid. In another embodiment, the mixture comprises at least two long chain omega-3 fatty acids. In another embodiment, the mixture comprises at least two short chain omega-3 fatty acids.

In one preferred embodiment, the mixture comprises at least two omega-3 fatty acids selected from the group consisting of eicosapentaenoic acid, docosahexaenoic acid, stearidonic acid, and linolenic acid.

Another presently preferred embodiment features a mixture of oils wherein the constituent oils contain either or both of short chain omega-3s and long chain omega-3s. In this aspect of the invention, the mixture contains at least two different oils. The omega-3 fatty acid containing oil mixtures may be prepared by first obtaining the constituent oils by either extracting them from the natural sources using methods known to persons skilled in the art, or by purchasing the oils from commercial sources. Second, the constituent oils may be blended by simply combining and mixing the oils using methods known to persons skilled in the art. The quantity of each constituent oil to be added when preparing the mixture of this invention is calculated based on the desired concentration of a particular omega-3 in the mixture. Optionally, any or all of the constituent oils, or the mixture of oils, or both, may be emulsified. In one embodiment, the mixture comprises at least one plant-derived oil, including but not limited to chia oil, echium oil, and flax oil, and at least one marine-derived oil including but not limited to fish oil and krill oil. In another embodiment, the mixture comprises at least two plant-derived oils. In another embodiment, the mixture comprises at least two marine-derived oils. In another embodiment, the mixture comprises at least one long chain omega-3 containing oil and at least one short chain omega-3 containing oil. In another embodiment, the mixture comprises at least two long chain omega-3 containing oils. In another embodiment, the mixture comprises at least two short chain omega-3 containing oils.

In one preferred embodiment, the mixture comprises at least two oils selected from the group consisting of chia oil, echium oil, flax oil, krill oil and fish oil.

In another preferred embodiment, the mixture comprises at least two oils selected from the group consisting of chia oil, echium oil, flax oil, fish oil, and krill oil, wherein each constituent oil is present in an amount ranging from 1% to 99% by weight of the total weight of the selected oils.

In yet another preferred embodiment, the mixture comprises at least two oils selected from the group consisting of chia oil, echium oil, flax oil, fish oil, and krill oil, wherein each constituent oil is present in an amount ranging from 10% to 90% by weight of the total weight of the selected oils,

In yet another preferred embodiment, the mixture comprises at least two oils selected from the group consisting of chia oil, echium oil, flax oil, fish oil, and krill oil, wherein each constituent oil is present in an amount ranging from 40% to 60% by weight of the total weight of the selected oils,

Embodiments of the invention that include emulsified oils and/or emulsified mixtures encompass both oil-in-water and water-in-oil emulsions. These emulsions are typically comprised of water, one or more oils, an emulsifier, and a stabilizer. Suitable emulsifiers include but are not limited to surfactants (anionic, cationic, or nonionic), proteins, and other food grade emulsifiers. Suitable emulsion stabilizers include but are not limited to glycerols. Emulsification may be performed according to methods known to persons skilled in the art. These methods include but are not limited to high-sheer mixing, mechanical mixing, and ultrasonic mixing. The emulsions of the present invention are preferably prepared to a median droplet size ranging from 0.1 μm-5 μm. In one embodiment, the emulsified omega-3 oils and mixtures are prepared to a median droplet size of 1.3 μm.

The present invention also includes formulations of the omega-3 containing mixtures of the invention. In this aspect of the invention, the compositions of the invention may be combined with one or more usual excipients, such as carriers, preservatives, sweeteners, flavors, etc., and/or other dietary supplements such as vitamins, minerals, antioxidants, herbs, fiber and the like. Alternatively, the individual omega-3 components may be combined first with one or more of such excipients, and then mixed to prepare the formulations of omega-3 mixtures of the present invention. The formulations of the present invention are those suitable for oral administration, parenteral administration, enteral administration (tube feeding), or for use as a dietary supplement. Formulations embodied by this aspect of the invention include but are not limited to liquids, tablets, capsules, gel caps, lozenges, fizzy tablets, leathers, chews, films or sheets suitable for oral administration, powders, emulsions, and food additives. Also included are formulations wherein the mixtures of the present invention are formulated as active pharmaceutical ingredients (both prescription and over-the-counter), or wherein the mixtures of the present invention are co-formulated with other active pharmaceutical ingredients. The formulations of this aspect of the invention may be prepared using any of a variety of formulation techniques known to persons skilled in the art.

Other embodiments of the invention are foods incorporating the mixtures of the invention. These foods can take a variety of forms including but not limited to nutritional beverages, baked goods, puddings, confections, snack foods, ice cream, frozen confections and novelties, and non-baked, extruded foods such as bars.

The present invention also includes methods of using the omega-3 mixtures of the invention. In this aspect of the invention, the omega-3 mixtures of the invention may be administered to human beings in suitable dosage forms, or incorporated into food products. The routes of administration include but are not limited to oral, parenteral, and enteral (feeding tube). In one embodiment, the mixtures of the invention are administered as dietary supplements so as to increase dietary intake of short chain omega-3s, or long chain omega-3s, or both. In this embodiment, the mixtures provide the subject with essential fats needed on a daily basis. In another embodiment, the mixtures of the invention are administered as a treatment for or to prevent a disease or adverse health condition including but not limited to cardiovascular disease, depression, senility, arthritis, premenstrual syndrome, dysmenorrheal, attention deficit hyperactivity disorder, and hyperlipidemia. In yet another embodiment of the invention, the mixtures of the invention are co-administered (e.g., simultaneously or sequentially) with an active pharmaceutical ingredient (including both prescription and over the counter drugs), or with other dietary supplements or other supplements, in either the same or separate formulations.

In one particularly preferred embodiment, mixtures of the present invention which comprise omega-3 oils are administered such that each constituent oil in the mixture is provided at a dosage of 0.01 mg to 50 g per day.

### EXAMPLES

The following examples further illustrate various aspects of the invention. These examples are merely illustrative and are not meant to limit the present invention in any way.

Examples 1 and 2 describe formulations useful for supplementing intake of omega-3s in healthy individuals. Examples 3-9 describe formulations useful for treating adverse health conditions.

### Example 1

### Vegetarian Formulation Useful As a Rich Source of Omega-3s

A formulation is prepared wherein each dosage form contains a mixture of 500 mg of echium oil and 500 mg of chia oil. This formulation can be administered preferably once or twice per day. This formulation is particularly useful for vegetarians because it provides more potential to make long-chain omega-3s (which are biologically active) than an equal weight of flax oil.

### Example 2

### Formulation Useful for Increased Omega-3 Absorption

A formulation is prepared wherein each dosage form contains a mixture of 750 mg of krill oil and 250 mg of fish oil. This formulation is particularly useful because it provides for increased absorption (due to the phospholipid omega-3s in the krill oil) of omega-3s relative to an equal weight of fish oil.

### Examples 3

### Formulations Useful for Improving Cognition and Attention

A formulation is prepared wherein each dosage form contains a mixture of 3.2 g fish oil (containing 50% EPA and 20% DHA) and 381 mg of plant-derived oil. This formulation may be useful for improving cognition and attention.

### Example 4

### Formulation Useful for Improving Cardiovascular Health

A formulation is prepared wherein each dosage form contains a mixture of 1.8 g fish oil (containing 1.06 g EPA and 54 mg DHA) and 1.4 g of a plant-derived oil (containing 591 mg of DHA). This formulation may be useful for improving cardiovascular health.

### Example 5

### Formulation Useful for Improving Blood Pressure

A formulation is prepared wherein each dosage form contains 7.5 g of flax oil (containing 4 g of LNA) and 6.3 g of chia oil (containing 4 g of LNA). This formulation may be useful for improving blood pressure.

### Example 6

### Formulation Useful for Improving Joint Health

A formulation is prepared wherein each dosage form contains a mixture of 1.54 g echium oil and 72 mg plant-derived oil (containing 30 mg of DHA). This formulation may be useful for improving joint health.

### Example 7

### Formulation Useful for Improving Mood and Depression

A formulation is prepared wherein each dosage form contains a mixture of 1.5 g fish oil (containing 900 mg EPA) and 1.8 g echium oil (containing 250 mg SA). This formulation may be useful for improving mood and alleviating depression.

### Example 8

### Formulation Useful for Improving Attention Deficit Hyperactivity Disorder

A formulation is prepared wherein each dosage form contains a mixture of krill oil (containing 170 mg EPA and 100 mg DHA) and 970 mg of fish oil (containing 388 mg EPA and 194 mg DHA). . This formulation may be useful for improving attention deficit hyperactivity disorder.

### Example 9

### Formulation Useful for Improving Cardiovascular Health and Premenstrual Syndrome

A formulation is prepared wherein each dosage form contains a mixture of 1 g krill oil (containing 170 mg EPA and 100 mg DHA) and 515 mg fish oil (containing 170 mg EPA and 113 mg DHA). This formulation should be taken eight days prior to and two days during menstruation each month.

In the manner described above, the present invention thus provides for mixtures of omega-3s as well as methods of preparing such and methods of using such. While this invention has been described with reference to specific embodiments, these are illustrative only. Modifications, such as the adaptation and optimization of certain parameters, will be apparent to those skilled in the art by studying the present specification. All references cited herein are incorporated by reference in their entireties.

What we claim is:

1. A composition comprising a long chain omega-3 fatty acid and a short chain omega-3 fatty acid, wherein the weight ratio of said long chain omega-3 fatty acid to said short chain omega-3 fatty acid is in the range of about 1:2 to about 1:10.

2. The composition of claim 1, wherein at least one of said long chain omega-3 fatty acid and said short chain omega-3 fatty acid is emulsified.

3. The composition of claim 1, wherein the weight ratio of said long chain omega-3 fatty acid to said short chain omega-3 fatty acid is in the range of about 1:2 to about 1:5.

4. A composition comprising a first component and a second component, wherein said first component comprises krill oil, and said second component is selected from the group consisting of fish oil, flax oil, chia oil, echium oil, and mixtures thereof.

5. The composition of claim 4 wherein at least one of said first component and said second component is emulsified.

6. A composition comprising a first component and a second component, wherein said first component comprises fish oil, and said second component is selected from the group consisting of chia oil, echium oil, and mixtures thereof.

7. The composition of claim 6 wherein at least one of said first component and said second component is emulsified.

8. A formulation comprising a first component, a second component, and an excipient, wherein said first component comprises a marine-derived omega-3 containing oil, and said second component comprises a plant-derived omega-3 containing oil.

9. The formulation of claim 8, wherein at least one of said first component and said second component is emulsified.

10. A formulation comprising a first component, a second component, and an excipient, wherein said first component comprises a long chain omega-3 fatty acid, and said second component comprises a short chain omega-3 fatty acid.

11. The formulation of claim 10, wherein at least one of said first component and said second component is emulsified.

12. The formulation of claim 10, wherein the weight ratio of said long chain omega-3 fatty acid to said short chain omega-3 fatty acid is in the range of about 2:1 to about 1:10.

13. The formulation of claim 10, wherein the weight ratio of said long chain omega-3 fatty acid to said short chain omega-3 fatty acid is in the range of about 1:0.8 to about 1:5.

14. A process for forming a composition comprising mixing at least two omega-3 fatty acids selected from the group consisting eicosapentaenoic acid, docosahexaenoic acid, linolenic acid and stearidonic acid.

15. The process of claim 14, further comprising emulsifying at least one omega-3 fatty acid prior to mixing.

16. A food or beverage comprising the composition of claim 1.

17. A food or beverage comprising the composition of claim 4.

18. A food or beverage comprising the composition of claim 6.

19. A food or beverage comprising the formulation of claim 8.

20. A food or beverage comprising the formulation of claim 10.

21. A method of increasing absorption of dietary omega-3s in a subject, comprising administering to said subject the composition of claim 1.

22. A method of increasing absorption of dietary omega-3s in a subject, comprising administering to said subject the composition of claim 4.

23. A method of increasing absorption of dietary omega-3s in a subject, comprising administering to said subject the composition of claim 6.

24. A method of increasing absorption of dietary omega-3s in a subject, comprising administering to said subject the formulation of claim 8.

25. A method of increasing absorption of dietary omega-3s in a subject, comprising administering to said subject the formulation of claim 10.

26. A method of treating a condition selected from the group consisting cardiovascular disease, hypertension, joint disease, mood disorders, depression, cognition-related disorders, attention deficit hyperactivity disorder, and premenstrual syndrome, said method comprising administering to a subject, the composition of claim 1.

27. A method of treating a condition selected from the group consisting cardiovascular disease, hypertension, joint disease, mood disorders, depression, cognition-related disorders, attention deficit hyperactivity disorder, and premenstrual syndrome, said method comprising administering to a subject, the composition of claim 4.

28. A method of treating a condition selected from the group consisting cardiovascular disease, hypertension, joint disease, mood disorders, depression, cognition-related disorders, attention deficit hyperactivity disorder, and premenstrual syndrome, said method comprising administering to a subject, the composition of claim 6.

29. A method of treating a condition selected from the group consisting cardiovascular disease, hypertension, joint disease, mood disorders, depression, cognition-related disorders, attention deficit hyperactivity disorder, and premenstrual syndrome, said method comprising administering to a subject, the formulation of claim 8.

30. A method of treating a condition selected from the group consisting cardiovascular disease, hypertension, joint disease, mood disorders, depression, cognition-related disorders, attention deficit hyperactivity disorder, and premenstrual syndrome, said method comprising administering to a subject, the formulation of claim 10.
